SEQUENCE LISTING (14 sequences)

SEQ ID NO: 1 - protein, 128 aa, Unknown
  sPD-1 AA
MHHHHHHLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVT

SEQ ID NO: 2 - DNA, 396 bp, Unknown
  sPD-1 NT
catatgcatcatcatcaccaccacctagaggtccccaatgggccctggaggtccctcaccttctacccagcctggctcacagtgtcagagggagcaaatgccaccttcacctgcagcttgtccaactggtcggaggatcttatgctgaactggaaccgcctgagtcccagcaaccagactgaaaaacaggccgccttctgtaatggtttgagccaacccgtccaggatgcccgcttccagatcatacagctgcccaacaggcatgacttccacatgaacatccttgacacacggcgcaatgacagtggcatctacctctgtggggccatctccctgcaccccaaggcaaaaatcgaggagagccctggagcagagctcgtggtaacataaatcgat

SEQ ID NO: 3 - protein, 182 aa, Unknown
  Homo sapiens
TTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 4 - protein, 181 aa, Unknown
  Mus musculus
TTASPSQVRQNYHQDAEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDES

SEQ ID NO: 5 - protein, 181 aa, Unknown
  Rattus norvegicus
TTASPSQVRQNYHQDSEAAINRQINLELYASYVYLSMSCYFDRDDVALKNFAKYFLHQSHEEREHAEKLMKLQNQRGGRIFLQDIKKPDRDDWESGLNAMRCALHLEKSVNQSLLELHKLATDKNDPHLCDFIETHYLNEQVKSIKELGDHVTNLRKMGAPESGMAEYLFDKHTLGHGDES

SEQ ID NO: 6 - protein, 170 aa, Unknown
  Ovis aries
TTASPSQVRQNYHQDSEAAINRQINLELYASYVYLSMSYYFDRDDVALKNFAKYFLHQSHEEREHAERLMKLQNQRGARIFLQDIKKPDRDDWENGLNAMECALCLERSVNQSLLELHKLATEKNDPHLCDFIETHYLNEQVEAIKELGDHITNLRKMGALWIGHGRVPL

SEQ ID NO: 7 - protein, 180 aa, Unknown
  Sus scrofa
TTSCSSQVRQNYHQDSEAAINRQINLELYASYVYLSMSYYFDRDDVALKNFAKYFLHQSHGGRGHAEKLMKLQTQRGARIFLQDIMKPERDDWENGLTAMEFALHVVKNVYQSLLELHKLATDKNDPHLCDFIETHYLHEQVKAIKELGDHITNLHRMGAPEYGMAEYLFDKHTLGSSES

SEQ ID NO: 8 - protein, 185 aa, Unknown
  Cricetulus griseus
TTTALTTASPSQVRQNYHQDSEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETHYLNEQVKSIKELGDHVTNLRKMGAPEAGMAEYLFDKHTLGHSES

SEQ ID NO: 9 - protein, 182 aa, Unknown
  Macaca mulatta
TTASTSQVRQNYHQDSEAAINRQINLELYASYVYLSMSYYFDRDDVALKNFAKYFLHQSHEEREHAEKLMKLQNQRGGRIFLQDIKKPDYDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 10 - protein, 185 aa, Unknown
  Bos taurus
EPEPPLNPPKPIRQNYCPKCEATVNSHAALEFHASFQCLAMAFYLDCDDMALKHFSRFFLLCSHEHSERAENLLFLQNQRGGRTCFLDIRKPETQQRESGLQAMQDILHLEKCVNQSLLNLYQLATDSSDAHLCHFLETHHLDQQVKFIKELGYVSNLSNVESLEGSLAEYVFDKLTLGDGDKND

SEQ ID NO: 11 - protein, 182 aa, Unknown
  Gorilla gorilla
TTASTSQVRQNYHQDSEAAINSQINLELYASYVYLSMSYYFDRDDVALKNFAKYFLHQSHEEREHAEKLMKLQNQQGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 12 - protein, 182 aa, Unknown
  Pan paniscus
TTASTSQVRQNYHQDSEAAINRQINLELYASYVYLSMSYYFDRDDVALKNFAKYFLHQSHEEREHAEKLMELQNQLGGRIFLQDIKKPDCDDWESRLNAMECALHLEKNVNQSLLELHKLATDKNDPHLCDFIETHYLNEQVKAIKELGDQVTNLRKMGAPESGLAEYLFDKHTLGDSDNES

SEQ ID NO: 13 - protein, 182 aa, Unknown; identical to SEQ ID NO: 3
  Pan troglodytes

SEQ ID NO: 14 - protein, 31 aa, Artificial Sequence
  Linker
GSSGGSGSSGGSGGGDEADGSRGSQKAGVDE

(19) United States
(12) Patent Application Publication
(10) Pub. No.: US 2022/0409550 A1
(43) Pub. Date: Dec. 29, 2022

(54) PD-1-DECORATED NANOCAGES AND USES THEREOF

(71) Applicants: KOREA INSTITUTE OF SCIENCE AND TECHNOLOGY, Seoul (KR); KYUNGPOOK NATIONAL UNIVERSITY INDUSTRY-ACADEMIC COOPERATION FOUNDATION, Daegu (KR)

(72) Inventors: In-San KIM, Seoul (KR); Eun Jung LEE, Daegu (KR); Gi-hoon NAM, Seoul (KR); Gi-beom KIM, Seoul (KR); Hyo-Dong SUNG, Daegu (KR)

(73) Assignees: KOREA INSTITUTE OF SCIENCE AND TECHNOLOGY, Seoul (KR); KYUNGPOOK NATIONAL UNIVERSITY INDUSTRY-ACADEMIC COOPERATION FOUNDATION, Daegu (KR)

(21) Appl. No.: 17/843,418

(22) Filed: Jun. 17, 2022

(30) Foreign Application Priority Data
Jun. 28, 2021 (KR) 10-2021-0083980

(51) Int. Cl. A61K 9/51; C07K 14/79; A61P 35/00

(52) U.S. Cl. CPC A61K 9/5169; C07K 14/79; A61P 35/00; B82Y 5/00

(57) ABSTRACT

Provided are a programmed cell death protein 1 (PD-1)-decorated nanocage and use thereof. The PD-1-decorated nanocage (PdNC) of the present disclosure may block PD-1 and programmed cell death-ligand (PD-L) signaling and may induce anti-tumor immunity activation at two immune checkpoints of tumor microenvironment (TME) (effector phase) and tumor-draining lymph node (TDLN) (innate phase), thereby increasing the adaptability of PD-1 and PD-L blockade-based therapy. Accordingly, it may be applied to various kinds of cancer therapies.

### CROSS-REFERENCE TO RELATED APPLICATIONS

This application claims the benefit under 35 USC 119(a) of Korean Patent Application No. 10-2021-0083980 filed on Jun. 28, 2021, in the Korean Intellectual Property Office, the entire disclosure of which is incorporated herein by reference for all purposes.

### REFERENCE TO AN ELECTRONIC SEQUENCE LISTING

The contents of the electronic sequence listing (“NewApp_0210920002_SequenceListing_AsFiled.txt”; Size is 19 kilobytes, and it was created on Jun. 14, 2022) is herein incorporated by reference in its entirety.

### BACKGROUND OF THE INVENTION

### 1. Field of the Invention

The present disclosure relates to a programmed cell death protein 1 (PD-1)-decorated nanocage and use thereof.

### 2. Description of the Related Art

Immune checkpoint blockade-based therapies have shown considerable clinical benefits in cancer patients. In particular, programmed cell death protein 1 (PD-1) and programmed cell death-ligand (PD-L) blockade-based therapy has achieved remarkable clinical success for various cancers, such as non-small cell lung cancer, melanoma, and renal cell cancer. PD-1 is expressed on immune cells, mainly on the surfaces of the activated T cells, and PD-1 on the surface of T cells specifically binds with PD-L1 or PD-L2, resulting in immunosuppression. Tumor cells escape from the immune surveillance by over-expressing PD-L1 and PD-L2.

Accordingly, to interfere with PD-1 and the interaction of its ligands and to boost the immune response against cancer, monoclonal antibodies have been developed and have shown significant anti-tumor effects. However, despite the significant clinical benefit of PD-1 and PD-L blockade, the significant efficacy is observed in only a minority of patients, and thus there has been a limitation in its application to several cancers. Consequently, more effective therapeutics and strategies are needed in PD-1 and PD-L blockade-based therapy.

PD-1 and PD-L blockade-based therapy is commonly considered to exhibit the efficacy by reactivating T cells in the tumor microenvironment (TME). PD-L1 is expressed by immune cells, including antigen-presenting cells (APCs) such as dendritic cells (DCs). DCs are the most potent APCs, and they have a vital role in anti-tumor immunity by mediating priming, activation, and reactivation of T cells in lymphoid organs. According to recent reports, the action of PD-1 and PD-L blockades in tumor-draining lymph nodes (TDLNs) is crucial for effective cancer immunotherapy. It was demonstrated that the status of the PD-1 and PD-L1 expression in metastatic lymph nodes is associated with poor prognostic features. It has also been revealed that PD-L1 of DCs can have a critical role in the inhibition of T cells that are newly activated and re-activated. Therefore, significant attention is being paid to the development of an effective system for delivering PD-1 and PD-L blockades to both the TME and TDLNs that can inhibit T cell exhaustion and induce DC-mediated T cell activation and re-activation.

Meanwhile, nanoscale materials have been widely used in the medical field, such as in drug delivery, owing to their high surface and volume ratios and biofunctionalization ability with various specific molecules. In particular, their small size, which is the most important feature of nanomaterials, allows them to deliver efficiently bio-functional molecules for various target organs. It is also known that materials with an approximate size of 10-100 nm primarily enter the lymphatic vessels, whereas materials smaller than 10 nm are mainly absorbed through blood capillaries. It has been recently reported that a lymphatic-targeted delivery system using S-nitrosated nanoparticles (SNO-NPs) was developed, and it was demonstrated that mid-size (30 nm) SNO-NPs significantly increase the total lymph node (LN) accumulation via passive lymph drainage and penetration, as compared to small (10 nm) and large (100 nm) SNO-NPs. In another report, intratumoral injected melittin-NPs with sizes of 10-20 nm were developed, which were drained to LNs and activated the APCs, leading to systemic anti-tumor immune responses.

In view of this background, the present inventors genetically incorporated PD-1 into ferritin nanocages to prepare PD-1-decorated nanocages (PdNCs), which were found to exhibit excellent anti-cancer activity according to anti-tumor immunity activation at two checkpoint sites: TME (effector phase) and TDLN (innate phase), thereby completing the present disclosure.

### PRIOR ART DOCUMENT

### Non-Patent Document

(Non-Patent Document 1) 1. L. F. Sestito, S. N. Thomas, Biomaterials 265 (2021) 120411.

### SUMMARY OF THE INVENTION

An object of the present invention is to provide a pharmaceutical composition for preventing or treating cancer, the pharmaceutical composition including, as an active ingredient, nanocages formed by self-assembly of a fusion protein including a programmed cell death protein 1 (PD-1) and a self-assembling protein.

Another object of the present invention is to provide a method of preventing or treating cancer, the method including the step of administering, to an individual, the pharmaceutical composition including, as an active ingredient, the nanocages formed by self-assembly of the fusion protein including the programmed cell death protein 1 (PD-1) and the self-assembling protein.

Still another object of the present invention is to provide a protein nanocage formed by self-assembly of the fusion protein of the present disclosure.

Still another object of the present invention is to provide a drug delivery carrier including the nanocages of the present disclosure.

Still another object of the present invention is to provide a drug delivery system including the nanocages of the present disclosure.

### BRIEF DESCRIPTION OF THE DRAWINGS

FIG. 1 shows a schematic illustration of PdNCs that block programmed cell death protein 1 (PD-1)/programmed cell death-ligand (PD-L) signaling and induce anti-tumor immunity activation at two checkpoint sites of the tumor microenvironment (TME) (effector phase) and tumor-draining lymph node (TDLN) (innate phase), in which firstly, PdNCs can reactivate tumor-specific T cells within the TME, which can promote death of tumor cells and release of tumor antigens, and secondly, PdNCs can efficiently drain into lymphatic capillaries and TDLNs along with tumor antigen-specific DCs, which can induce activation of T cell through interfering between PD-1 on T cells and PDLs on DCs, and subsequently, the (re-)activated tumor-specific T cells traffic into the TMEs to kill the tumor cells;

FIG. 2 shows a schematic illustration of PdNCs biosynthesized into a self-assembled nano-scale cage-like structure by an E. coli expression system, in which FIG. 2A shows a schematic diagram showing a vector map (left) and a synthesis procedure (right) of the PdNCs in E. coli based on a 3D protein structure of hFTN (blue, PDB 3AJO) and ecto-domain of murine PD-1 (magenta, PDB 1NPU), wherein a flexible linker peptide (red, indicated as L) was inserted between hFTN and PD-1, FIG. 2B shows SDS-PAGE analysis of lysates for E. coli BL21(DE3) cells that were transformed with an empty vector (Ctrl) or plasmids encoding an indicated PdNCs (left) and PdNCs purified by nickel affinity chromatography (right), showing production of PdNCs (M, a protein molecular marker; Sol., a soluble fraction of the cell lysates; and Insol., an insoluble fraction of the cell lysates), and further, 38.7 kDa is a theoretically calculated molecular weight of PdNCs, FIG. 2C shows TEM images and FIG. 2D DLS analysis of the purified PdNCs, showing a spherical cage-like structure with a nanoscale size, wherein wtNC represents a wild type of ferritin nanocages;

FIG. 3 shows binding of PdNCs with tumors on the PD1/PDL axis, (A and B) CT26.CL25 cells (IFN-γ pre-treated or not) were incubated with indicated concentrations for each of the NCs and stained with anti-ferritin antibodies and Alexa 488 (green) antibodies, the binding of the NCs with the tumor cells was analyzed by performing flow cytometry or IF microscopy, in which FIG. 3A shows representative histogram images (upper) indicating the NC binding affinity according to the concentration, wherein the binding affinity is presented as a relative MFI to a control (lower) (n=4), and FIG. 3B shows representative images of binding with tumor cells and 40 nM NCs (green), wherein nuclei were counterstained with Hoechst (blue) (scale bar=50 μm) (n=3), and P values were analyzed by performing a one-way analysis of variance (ANOVA) and Tukey's post-hoc test (***p<0.001);

FIG. 4 shows up-regulation of PD-L1 or PD-L2 expression by IFN-γ in CT26.CL25 tumor cells, in which relative MFI levels of PD-L1 or PD-L2 are shown in CT26.CL25 cells (left) and histogram (right) after treatment with indicated amounts of IFN-γ, and a one-way ANOVA and Tukey's post-hoc test (***p<0.001) (n=3) were performed;

FIG. 5 shows that PdNCs exhibit improved antagonistic activity with highly enhanced binding kinetics against PD-L, in which FIG. 5A shows representative sensorgrams and summary of SPR analysis of affinity and kinetics of PdNCs or sPD-1 against immobilized PD-L1 and PD-L2 in dextran chips, PdNCs (2.5-500 nM) or sPD-1 (1-50 μM) were injected with two-point serial dilution, and the binding kinetics were derived by fitting the sensorgrams to a 1:1 binding model, K_{D}=k_{d}/kₐ, and FIG. 5B shows antagonistic activity of PdNCs or sPD-1 analyzed using a bioluminescent PD-1/PD-L1 blockade reporter bioassay, wherein the data are presented as the mean of RLU against a buffer control from NFAT-mediated luciferase expression through PD-1/PD-L1 blockade and TCR activation in Jurkat T cells treated with PdNCs or sPD-1 (n=3), and P values were analyzed by performing a one-way ANOVA and Tukey's post-hoc test (*p<0.05, **p<0.01);

FIG. 6 shows results of bioluminescent PD-1/PD-L1 blockade reporter bioassay, in which wtNC exhibited no antagonistic activity, the data are presented as the mean of relative light units (RLU) against the control from the NFAT-mediated luciferase expression through PD-1/PD-L1 blockade and TCR activation in Jurkat T cells treated with wtNC (n=3);

FIG. 7 shows that PdNCs efficiently target TDLN and enhance the anti-tumor immune response, in which FIG. 7A shows ex vivo images of TDLN at indicated times (1 hr, 6 hr, 18 hr, or 24 hr) after intratumoral injection of Cy5.5-labeled PdNCs, wtNCs, sPD-1, or free Cy5.5 (lower) and quantification of TDLN/tumor signal ratios (upper) (n=4-5), and FIGS. 7B to 7E show that TDLNs were resected and analyzed from mice at day 3 after injection of PdNCs, wtNCs, sPD-1, or a buffer (control), wherein FIG. 7B shows that expressions of CD40 and CD86 on CD11c+ DC were evaluated by flow cytometry, and the data are presented as the means of the relative MFI to the control (n=4-5), FIG. 7C shows that CD44 expressions in the CD8+ T cells were analyzed by flow cytometry and are presented as the relative MFI to the control (n=4-5), FIG. 7D shows that single cells in the TDLN were co-cultured with the gp70 or β-gal peptide for 24 hr, and the released IFN-γ was determined by ELISA (n=3-4), and FIG. 7E shows that cross-priming ability of DC was confirmed through IFN-γ ELISA by co-culturing of CD11c+ cells from TDLNs and CD8+ splenocytes (n=4-5), and P values were analyzed by performing one-way ANOVA and Tukey's post-hoc test or student's t-test (* p<0.05, ** p<0.01);

FIG. 8 shows that PdNCs inhibit tumor growth by CD8+ T cell activation-mediated immunity against tumors, in which FIG. 8A shows time-course change of the tumor volume for CT26.CL25-bearing BALB/c mice treated once with a buffer, sPD-1, wtNCs, or PdNCs (n=6-10), FIG. 8B shows that the activation of CD8+ cell from tumor tissues was evaluated at day 3 after injection of PdNCs, wtNCs, sPD-1, or the buffer (control), CD3+ CD45.2+ CD8+ T cells in TME were labeled with anti-ki67 or IFNγ antibodies, and percentages of the ki67+ or IFNγ+ positive populations were analyzed by performing flow cytometry (n=3-4), FIG. 8C shows representative CD8+ T cell infiltration images in the tumor tissues from the CT26.CL25-bearing mouse at day 15 after injection of PdNCs, wtNCs, sPD-1, or the buffer (control), FIG. 8D shows quantification of infiltrating the CD8+ T cells in the tumor sections; these were analyzed from the fluorescence images including those in (C), the number of CD8+ cells/mm² was calculated using Image J software (n=3-5, different fields for each image), FIG. 8E shows that DC infiltration from tumor tissue was evaluated at day 3 after injection of PdNCs, wtNCs, sPD-1, or the buffer (control), and percentages of CD45.2+ CD11c+ cells in TME were detected with flow cytometry analysis (n=4-5), and FIG. 8F shows that the tumor-free mice from (A) were re-injected with 1×10⁶ tumor cells after four weeks in the opposite site of the primary tumor (n=3), and a one-way ANOVA and Tukey's post-hoc test were performed (* p<0.05, ** p<0.01, *** p<0.001);

FIG. 9 shows body weights of the resected tumors after completion of the experiment of FIG. 8A, wherein a one-way ANOVA and Tukey's post-hoc test were performed (*** p<0.001) (n=6-10);

FIG. 10 shows changes in the body weights of CT26.CL25 tumor mouse models treated with the buffer, sPD-1, wtNCs, or PdNCs (n=6-10); and

FIG. 11 shows results of H&E staining for measuring potent toxicity of the buffer (PBS), sPD1, wtNCs, and PdNCs in indicated organs (n=2-5, different fields for each image).

### DETAILED DESCRIPTION OF THE PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

One aspect of the present disclosure provides a pharmaceutical composition for preventing or treating cancer, the pharmaceutical composition including, as an active ingredient, nanocages formed by self-assembly of a fusion protein including a programmed cell death protein 1 (PD-1) and a self-assembling protein.

As used herein, the term “immune checkpoint blockade” refers to inhibition of immune responses produced by specific types of immune cells, such as T lymphocytes, and some cancer cells, and blockade or inhibition of a specific protein that prevents T lymphocytes from killing cancer cells. When the specific protein is blocked, immune cells such as tumor-specific T cells are able to better kill cancer cells. The immune checkpoint which has been known so far includes programmed cell death protein 1 or its ligand PD-L1/PD-L2, or CTLA-4/B7-1/B7-2, etc.

As used herein, the term “programmed cell death protein 1 (PD-1)” is expressed on immune cells, mainly, on the surfaces of activated T cells, and PD-1 on T cells specifically binds to a programmed cell death-ligand (PD-L), PD-L1 or PD-L2 to induce immune suppression. Tumor cells are known to escape from immune surveillance by over-expressing PD-L1 and PD-L2.

In the present disclosure, the PD-1 may be, but is not particularly limited to, murine-derived PD-1, specifically, a monomeric form of soluble PD-1 (sPD-1) which is an ecto-domain of murine PD-1.

The PD-1 may include an amino acid sequence of SEQ ID NO: 1.

The amino acid sequence of SEQ ID NO: 1 may be obtained from NIH GenBank which is a public database. In the present disclosure, the amino acid sequence of SEQ ID NO: 1 may include an amino acid sequence having at least 70%, 75%, 76%, 85%, 90%, 95%, 96%, 97%, 98%, 99%, 99.5%, 99.7%, or 99.9% or more homology or identity to the amino acid sequence represented by SEQ ID NO: 1. Additionally, it is apparent that any amino acid sequence, in which part of the sequence is deleted, modified, substituted, or added, may also fall within the scope of the present disclosure, as long as the amino acid sequence has such a homology or identity and exhibits efficacy corresponding to that of the protein including the amino acid sequence of SEQ ID NO: 1.

For example, it may be a case where the N-terminus, the C-terminus, and/or inside of the amino acid sequence is added with a sequence that does not alter the function of the protein, or deleted, or has a naturally occurring mutation, a silent mutation thereof, or a conservative substitution.

As used herein, the term “conservative substitution” refers to substitution of an amino acid with another amino acid having similar structural and/or chemical properties. Such amino acid substitution may generally occur based on similarity of polarity, charge, solubility, hydrophobicity, hydrophilicity, and/or amphipathic nature of a residue.

As used herein, the term “homology” or “identity” refers to a degree of similarity between two given amino acid sequences or nucleotide sequences, and may be expressed as a percentage. The terms homology and identity may be often used interchangeably with each other.

The sequence homology or identity of conserved polynucleotide or polypeptide may be determined by standard alignment algorithms and may be used with a default gap penalty established by the program being used. Substantially, homologous or identical sequences may generally hybridize to all or a part of the sequences under moderate or highly stringent conditions. It is also obvious that hybridization also includes hybridization with polynucleotides containing common codons or codons in consideration of codon degeneracy in polynucleotides.

Whether or not any two polynucleotide or polypeptide sequences have homology, similarity, or identity may be determined by a known computer algorithm such as the “FASTA” program as in Pearson et al. (1988) Proc. Natl. Acad. Sci. USA 85:2444 using default parameters. Alternatively, it may be determined by the Needleman-Wunsch algorithm (Needleman and Wunsch, 1970, J. Mol. Biol. 48:443-453), which is performed using the Needleman program of the EMBOSS package (EMBOSS:The European Molecular Biology Open Software Suite, Rice et al., 2000, Trends Genet. 16:276-277) (version 5.0.0 or later versions) (GCG program package (Devereux, J., et al., Nucleic Acids Research 12:387 (1984)), BLASTP, BLASTN, FASTA (Atschul, S. F., et al., J MOLEC BIOL 215:403 (1990); Guide to Huge Computers, Martin J. Bishop, ed., Academic Press, San Diego, 1994, and CARILLO et al. (1988) SIAM J Applied Math 48:1073). For example, the homology, similarity, or identity may be determined using BLAST or ClustalW of the National Center for Biotechnology Information.

The homology, similarity, or identity of polynucleotides or polypeptides may be determined by comparing sequence information using, for example, the GAP computer program, such as Needleman et al. (1970), J Mol Biol. 48:443, as disclosed in Smith and Waterman, Adv. Appl. Math (1981) 2:482. Briefly, the GAP program defines the homology, similarity, or identity as a value obtained by dividing the number of similarly aligned symbols (i.e., nucleotides or amino acids) by the total number of the symbols in the shorter of the two sequences. Default parameters for the GAP program may include (1) a binary comparison matrix (containing a value of 1 for identities and 0 for non-identities) and the weighted comparison matrix of Gribskov et al. (1986) Nucl. Acids Res. 14:6745, as disclosed in Schwartz and Dayhoff, eds., Atlas Of Protein Sequence And Structure, National Biomedical Research Foundation, pp. 353-358 (1979) (alternatively, a substitution matrix of EDNAFULL (EMBOSS version of NCBI NUC4.4); (2) a penalty of 3.0 for each gap and an additional 0.10 penalty for each symbol in each gap (or a gap opening penalty of 10 and a gap extension penalty of 0.5); and (3) no penalty for end gaps.

As used herein, the term “nanocage (NC)” refers to a hollow nanoparticle, and may include an inorganic nanocage and an organic nanocage. The inorganic nanocage is a hollow metal nanoparticle produced by reacting a metal nanoparticle with a different metal in boiling water, for example, a hollow gold (Au) nanoparticle produced by reacting silver (Ag) nanoparticle with chloroauric acid (HAuCl₄) in boiling water. The organic nanocage includes a protein nanocage which is a nanocage produced by self-assembly of a self-assembling protein.

In the present disclosure, the nanocage is an organic nanocage produced by self-assembly of a self-assembling protein, i.e., a protein nanocage.

As used herein, the term “self-assembling protein” refers to a protein capable of forming nanoparticles by forming multimers by regular arrangement at the same time as expression without the aid of a particular inducer. Examples of the self-assembling proteins include ferritin, small heat shock protein (sHsp), vault, P6HRC1-SAPN, M2e-SAPN, MPER-SAPN, various virus capsid proteins or bacteriophage capsid proteins, etc. Such self-assembling proteins are well described in Hosseinkhani et al. (Chem. Rev., 113(7):4837-4861, 2013). This document is incorporated herein by reference in its entirety.

The virus capsid protein and the bacteriophage capsid protein may be any one or more selected from the group consisting of a bacteriophage MS2 capsid protein, a bacteriophage P22 capsid protein, a Qβ bacteriophage capsid protein, a CCMV capsid protein, a CPMV capsid protein, an RCNMV capsid protein, an ASLV capsid protein, an HCRSV capsid protein, an HJCPV capsid protein, a BMV capsid protein, an SHIV capsid protein, an MPV capsid protein, an SV40 capsid protein, an HIV capsid protein, an HBV capsid protein, an adenovirus capsid protein, and a rotavirus VP6 protein, but are not limited thereto.

In the present disclosure, the self-assembling protein may be ferritin.

In the present disclosure, the self-assembling protein, ferritin may be any one or more selected from a ferritin heavy chain protein and a ferritin light chain protein, specifically, a ferritin heavy chain protein, and more specifically, a human-derived ferritin heavy chain protein, but is not limited thereto.

The ferritin may include any one or more amino acid sequences selected from the group consisting of SEQ ID NOS: 3 to 13, and may specifically include an amino acid sequence of SEQ ID NO: 3, but is not limited thereto.

In the present disclosure, the PD-1 and the self-assembling protein may be linked via a linker.

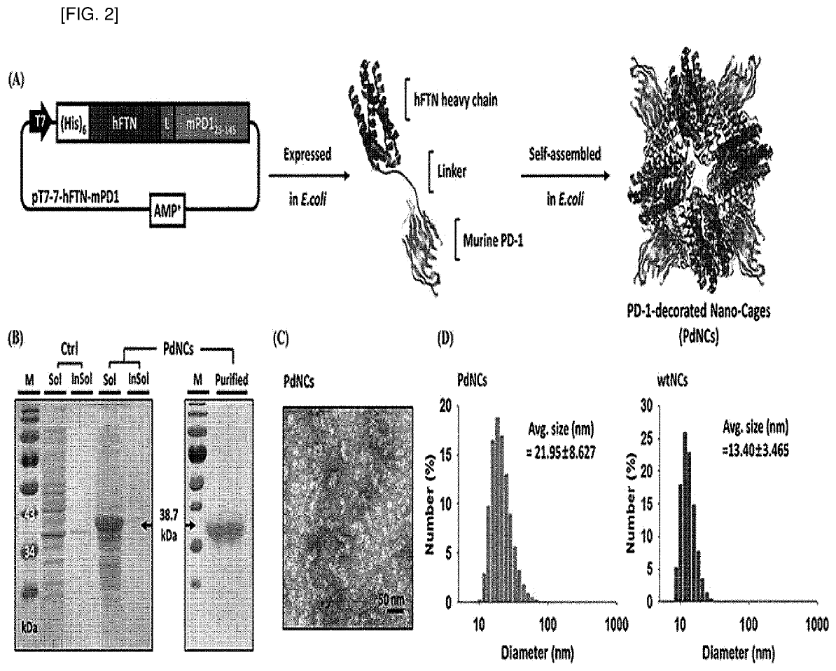

Specifically, the ecto-domain of the PD-1 capable of binding to PD-L1 and PD-L2 may be genetically integrated into the C-terminus of ferritin heavy chain subunit with the linker (FIG. 2A). Conformational flexibility of the C-terminal E-helix of ferritin subunit and the flexible glycine-rich linker were expected to provide sufficient accessibility of the ligands' binding capabilities.

The linker may include an amino acid sequence of SEQ ID NO: 14, but is not limited thereto.

The nanocage of the present disclosure may be formed by self-assembly of the fusion protein including the PD-1 and the self-assembling protein to present PD-1 on the surface thereof with high density.

The nanocage may be used interchangeably with PD-1-decorated ferritin nanocage, PD-1-decorated nanocage, PdNC, etc.

The nanocage of the present disclosure may be decorated with PD-1 with high density on the surface thereof, and thus its binding ability to PD-L1 and PD-L2-expressed cancer cells may be increased, as compared with a ferritin nanocage not decorated with PD-1, and sPD-1.

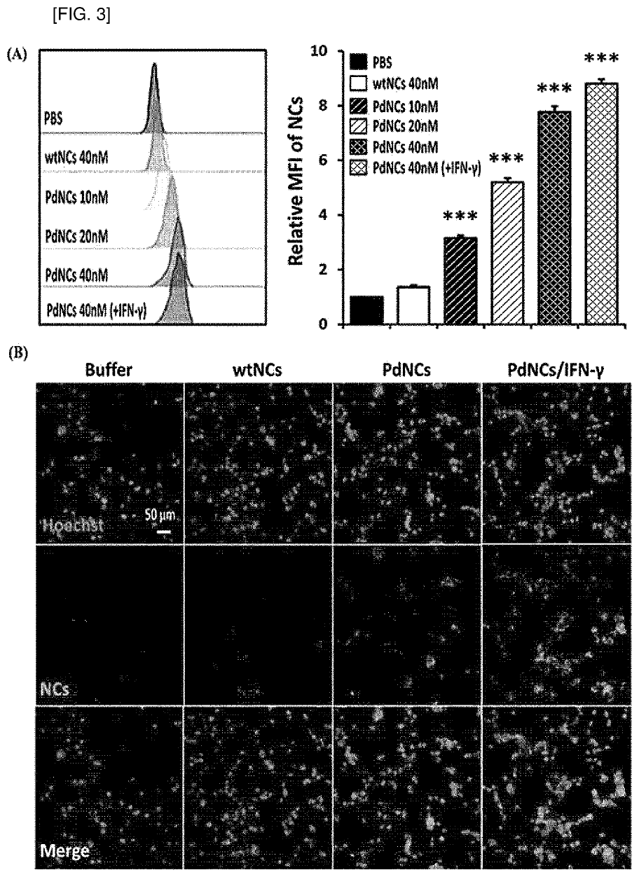

In one embodiment of the present disclosure, ferritin nanocage not decorated with PD-1 (wtNC) showed binding ability to CT26.CL25 cells through transferrin receptors (TfR) (1.29 times a buffer control), whereas PdNCs were more efficiently bound to CT26.CL25 cells in a concentration-dependent manner (8.80 times the buffer control), as compared to wtNC (FIG. 3A). It was confirmed that the amount of PdNCs bound to the PD-L1- and PD-L2-overexpressed CT26.CL25 cells was up-regulated, indicating that the binding of PdNCs to the tumor cells may be increased even more for in vivo tumor microenvironment conditions.

In fluorescence microscopic images, CT26.CL25 cells treated with PdNCs also showed more fluorescence than that of wtNCs (FIG. 3B).

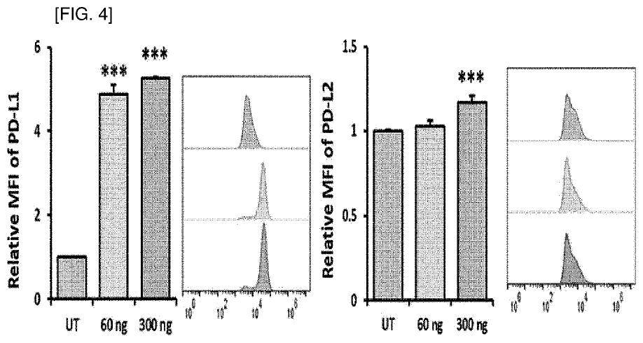

It is known that PD-L expression on the tumor cell surfaces is often upregulated by IFN-γ within tumor microenvironment. Consistently, the expression levels of PD-L1 and PD-L2 were significantly increased on the surface of the tumor cells treated with IFN-γ (FIG. 4). In a similar context, binding of PdNCs was significantly increased in the IFN-γ-treated cells.

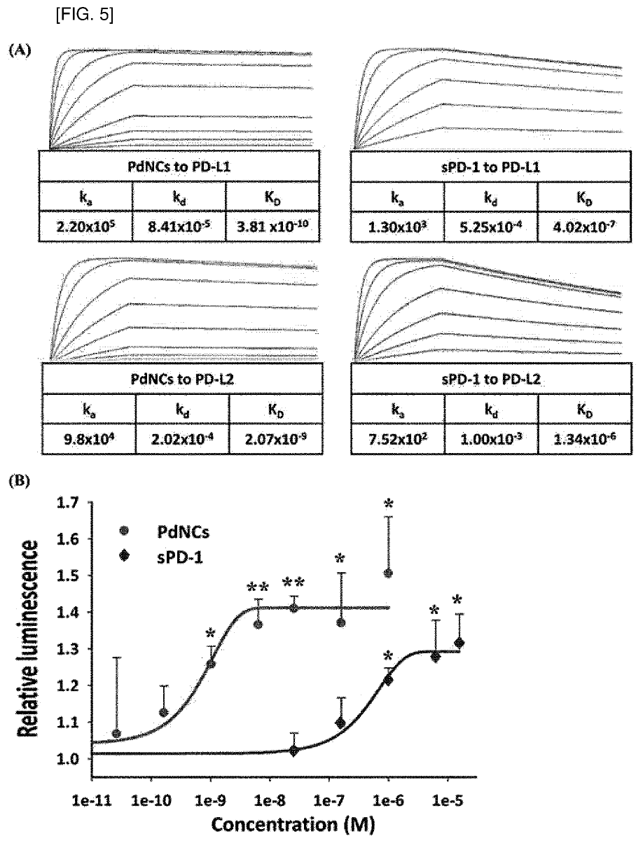

In another embodiment of the present disclosure, both PdNCs and sPD-1 were bound to PD-L1 and PD-L2 in a concentration-dependent manner, and sPD-1 bound to PD-L1 and PD-L2 has a low affinity (FIG. 5A). PdNCs were bound to PD-L1 and PD-L2 with nanomolar and sub-nanomolar affinities. Higher association rates (kₐ) and lower dissociation rates (kₐ) of PdNCs than sPD-1 were observed for both ligands, and an equilibrium dissociation constant (K_{D}) for PdNCs was decreased by 1057 times for PD-L1 and 647 times for PD-L2 in comparison to sPD-1. Thus, PD-1 on the surfaces of PdNCs is readily recognized by their ligands with an enhanced avidity effect.

These results suggest that the PD-1-decorated ferritin nanocage of the present disclosure has the increased binding ability to PD-L1 and PD-L2-expressed cancer cells, as compared with the ferritin nanocage not decorated with PD-1, and sPD-1.

The nanocage of the present disclosure may bind to PD-L1 and PD-L2-expressed cancer cells to improve antagonistic activity.

Further, the nanocage of the present disclosure may block any one or more signals selected from a programmed cell death protein 1 and a programmed cell death-ligand.

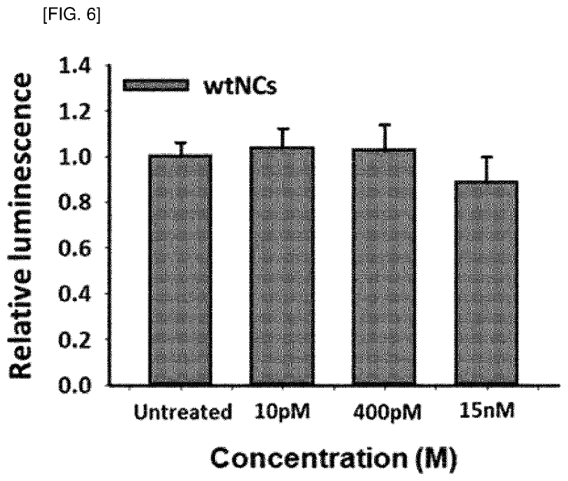

In one embodiment of the present disclosure, CHO-K1 cells that expressed murine PD-L1 and a protein that was designed to activate cognate TCRs were treated with PdNCs, sPD-1, or wtNCs. Continuously, Jurkat cell lines expressing PD-1, TCR, and nuclear factor activated T cell (NFAT)-inducible luciferase, which replaced primary T cells, were added and co-cultured for 6 hours. As a result, treatment of Jurkat T cells with low dosage PdNCs successfully increased TCR activation and NFAT-mediated luciferase expression through PD-1/PD-L1 binding (FIG. 5B). There was no signaling change for the wtNC-treated Jurkat T cells (FIG. 6), whereas the PdNC- and sPD-1-treated cells exhibited dose-dependent TCR activation signaling.

Meanwhile, there was substantial increase in the NFAT-mediated luciferase expression in the Jurkat T cell that was observed with treatment of a higher sPD-1 dosage. In particular, PdNCs exhibited a half maximal effective concentration (EC50) of 761.3 pM, which is 624 times higher than that of sPD-1 (457.3 nM).

Consequently, the PD-1-decorated ferritin nanocages of the present disclosure substantially improved their recognition with their enhanced affinity, supporting that PdNCs may serve as promising antagonistic agents for anti-cancer immunotherapy.

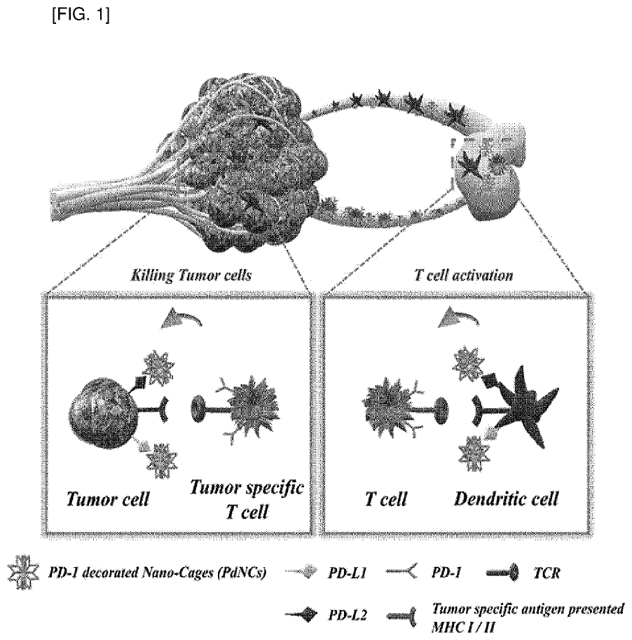

The nanocage of the present disclosure may induce anti-tumor immunity activation at two immune checkpoints of effector phase and innate phase (FIG. 1).

The anti-tumor immunity activation may be dendritic cell (DC)-mediated tumor-specific T cell activation. DCs are the most potent antigen-presenting cells (APCs), and they mediate priming, activation, and reactivation of T cells in lymphoid organs to activate tumor-specific T cells, contributing to cancer cell death.

The anti-tumor immunity activation at the effector phase may occur in the tumor microenvironment (TME).

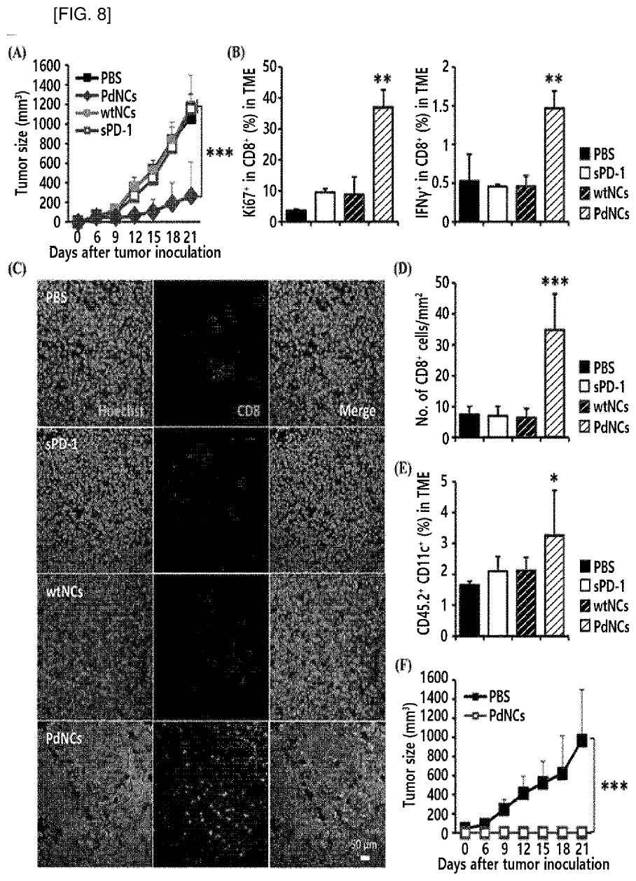

In one embodiment of the present disclosure, PdNC treatment increased ki67 and IFN-γ expression in CD3+ CD45.2+ CD8+ T cells in the TME, in which ki67 and IFN-γ are markers for the proliferation and effector functions of T cells (FIG. 8B), and the PdNC-treated group induced a significant increase in the tumor-infiltrating CD8+ T cells and DCs in comparison to other groups (FIGS. 8C to 8E). In particular, a significant increase of tumor-infiltrating CD8+ T cells was observed in the PdNC-treated mice (4.64 times a buffer control) (FIG. 8D).

These results suggest that activation of T cell immunity in TME is induced by the nanocage of the present disclosure.

The anti-tumor immunity activation at the innate phase may occur in the tumor-draining lymph node (TDLN).

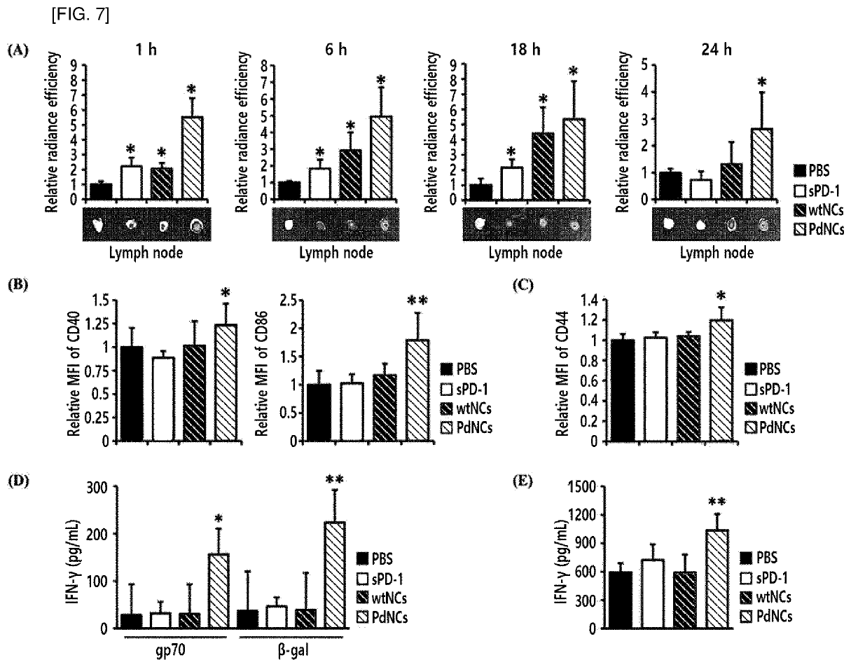

In one embodiment of the present disclosure, intratumorally injected PdNCs were rapidly drained and accumulated in TDLNs after 1 hr post injection (FIG. 7A). After 6 hr and 18 hr post injection, although the fluorescence intensity in the TDLNs of the wtNC-treated mice gradually increased due to its size, the fluorescence intensity in the TDLNs of the PdNC-treated mice are the highest at all time-points. In particular, at 24 hr, a stronger fluorescence intensity in the TDLNs of the PdNC-treated mice was observed than in any other group.

Further, PdNC treatment induced increases in the co-stimulatory molecules (CD40 and CD86) on the DCs in the TDLNs, enhancing DC maturation (FIG. 7B), and PdNC treatment increased CD44+ in the CD8+ T cells, wherein CD44+ is a marker for antigen-experienced T cells (FIG. 7C).

Moreover, PdNC treatment significantly increased IFN-γ secretion (FIGS. 7D to 7E).

These results indicate that the nanocage of the present disclosure efficiently reached and accumulated in the TDLNs, and may efficiently block the PD-1/PD-Ls pathway to improve DC maturation and T cell activation, and may potentiate activated DC-mediated anti-tumor T cell immune responses in TDLNs.

The nanocage of the present disclosure may exhibit tumor growth-inhibitory activity.

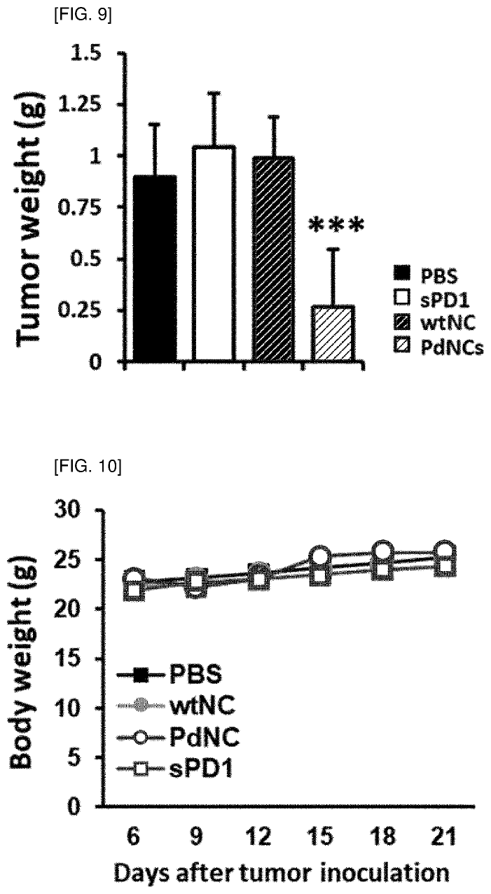

In one embodiment of the present disclosure, the PdNC-treated group dramatically reduced the tumor growth in comparison to other groups (FIG. 8A and FIG. 9). The PdNCs suppressed the tumor volumes by 75%, whereas 24 times higher molar doses of sPD-1 resulted in no significant reduction of the tumor volume. Furthermore, complete tumor regression was observed in approximately 33% (3 of 9) of the PdNC-treated group with only a single injection.

These results suggest that the nanocage of the present disclosure has tumor growth-inhibitory activity.

The nanocage of the present disclosure may form an immunologic memory.

In one embodiment of the present disclosure, when the PdNC-treated tumor-free mice were re-injected with the same tumor cells on day 21 after CT26.CL25 tumor inoculation into the CT26.CL25 mouse model, there was no tumor growth (FIG. 8F).

This result indicates that an immunologic memory was formed by the nanocage of the present disclosure.

The nanocage of the present disclosure may exhibit no in vivo toxicity.

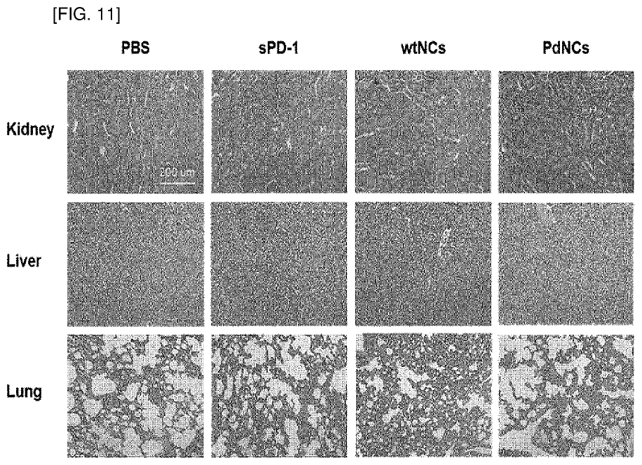

In one embodiment of the present disclosure, there was no decrease in the body weight of the mice of all groups, including in those treated with PdNCs, and there were no significant differences between the groups (FIG. 10). In addition, H&E stained images of major organs including liver, lung, and kidney from the PdNC-treated mice exhibited no differences compared to those in the control group (FIG. 11).

This result indicates no significant toxicity due to the nanocage of the present disclosure.

As described above, the present disclosure is significant in that the PD-1-decorated nanocage (PdNC) was prepared, which may block PD-1 and PD-L signaling and may induce anti-tumor immunity activation at the two immune checkpoint sites of TME (effector phase) and TDLN (innate phase), thereby increasing the adaptability of PD-1 and PD-L blockade-based therapy.

The fusion protein of the present disclosure may further include a tag peptide for purification at the N-terminus or C-terminus for efficient purification thereof. The tag peptide may include, for example, a His×6 peptide, a GST peptide, a FLAG peptide, a streptavidin binding peptide, a V5 epitope peptide, a Myc peptide, an HA peptide, etc.

Another aspect of the present disclosure provides a method of preventing or treating cancer, the method including the step of administering, to an individual excluding humans, the pharmaceutical composition including, as an active ingredient, the nanocages formed by self-assembly of the fusion protein including the programmed cell death protein 1 (PD-1) and the self-assembling protein.

The terms used herein are the same as described above.

The composition of the present disclosure may have use of “prevention” and/or “treatment” of cancer.

For prophylactic use, the composition of the present disclosure may be administered to an individual who has the disease, disorder, or condition described herein or is suspected of being at risk of developing the disease. For therapeutic use, the composition of the present disclosure may be administered to an individual such as a patient already suffering from a disorder described herein in an amount sufficient to treat or at least partly stop symptoms of the diseases, disorders, or conditions described herein. The amount effective for this use will vary according to the severity and course of the disease, disorder, or condition, previous treatment, a subject's health condition and responsiveness to a drug, and the judgment of physicians or veterinarians.

In the present disclosure, the cancer may include any cancer known in the art without limitation, for example, lung cancer (e.g., non-small cell lung cancer, small cell lung cancer, malignant mesothelioma), mesothelioma, pancreatic cancer (e.g., pancreatic duct cancer, pancreatic endocrine tumor), pharyngeal cancer, laryngeal cancer, esophageal cancer, gastric cancer (e.g., papillary adenocarcinoma, mucous adenocarcinoma, glandular squamous cell carcinoma), duodenal cancer, small intestine cancer, large intestine cancer (e.g., colon cancer, rectal cancer, anal cancer, familial colon cancer, hereditary nasal polyposis colon cancer, gastrointestinal interstitial tumor), breast cancer (e.g., invasive ductal cancer, non-invasive ductal cancer, inflammatory breast cancer), ovarian cancer (e.g., epithelial ovarian carcinoma, extra-testicular germ cell tumor, ovarian germ cell tumor, ovarian hypomalignant tumor), testicular tumor, prostate cancer (e.g., hormone-dependent prostate cancer, hormone-independent prostate cancer), liver cancer (e.g., hepatocellular carcinoma, primary liver cancer, extrahepatic bile duct cancer), thyroid cancer (e.g., medullary thyroid carcinoma), kidney cancer (e.g., renal cell carcinoma, transitional epithelial carcinoma of the renal pelvis and ureter), cervical cancer (e.g., cervical cancer, uterine body cancer, uterine sarcoma), brain tumors (e.g., medulloblastoma, glioma, pineal gonadoblastoma, spheroid gonadocytoma, diffuse gonadoblastoma, degenerative gonadoblastoma, pituitary adenoma), retinoblastoma, skin cancer (e.g., basal cell carcinoma, malignant melanoma), sarcoma (e.g., rhabdomyosarcoma, leiomyosarcoma, soft tissue sarcoma), malignant bone tumor, bladder cancer, blood cancer (e.g., multiple myeloma, leukemia, malignant lymphoma, Hodgkin's disease, chronic myelogenous disease), primary unknown cancer, etc.

The composition of the present disclosure may be included as an active ingredient in a pharmaceutical composition for preventing or treating cancer. The pharmaceutical composition may further include an appropriate carrier, excipient, or diluent commonly used in the preparation of the pharmaceutical composition. In this regard, the amount of the nanocage of the present disclosure, which is an active ingredient included in the pharmaceutical composition, may be, but is not particularly limited to, 0.1% by weight to 90% by weight, specifically, 1%) by weight to 50% by weight, based on the total weight of the composition.

The pharmaceutical composition may have any one formulation selected from the group consisting of tablets, pills, powders, granules, capsules, solution for internal use, syrups, sterile aqueous solutions, non-aqueous solvents, suspensions, emulsions, lyophilized agents, and suppositories, and may have various oral or parenteral formulations. When formulated, the formulation may be prepared by using diluents or excipients, such as a filler, an extender, a binder, a wetting agent, a disintegrating agent, a surfactant, etc., which are generally used. A solid formulation for oral administration includes a tablet, a pill, a powder, a granule, a capsule, etc., and the oral formulation may further include a pharmaceutically acceptable additive, e.g., a diluent, a binder, a swelling agent, a lubricant, etc. Further, lubricants such as magnesium stearate, talc, etc. may be used, in addition to simple excipients.

The diluent may include, but is not particularly limited to, for example, lactose, dextrin, mannitol, sorbitol, starch, microcrystalline cellulose, calcium hydrogen phosphate, anhydrous calcium hydrogen phosphate, calcium carbonate, sugars, etc.

The binder may include, but is not particularly limited to, for example, polyvinylpyrrolidone, kopovidone, gelatin, starch, sucrose, methyl cellulose, ethyl cellulose, hydroxyethyl cellulose, hydroxypropyl cellulose, hydroxypropyl alkyl cellulose, etc.

The swelling agent may include any one or more components selected from the group consisting of crosslinked polyvinylpyrrolidone, crosslinked sodium carboxymethyl cellulose, crosslinked calcium carboxymethyl cellulose, crosslinked carboxymethyl cellulose, sodium starch glycolate, carboxymethyl starch, sodium carboxymethyl starch, a potassium methacrylate-divinylbenzene copolymer, amylose, cross-linked amylose, starch derivatives, microcrystalline cellulose and cellulose derivatives, cyclodextrin and dextrin derivatives.

The lubricant may include, but is not particularly limited to, for example, stearic acid, stearic acid salt, talc, corn starch, carnauba wax, light anhydrous silicic acid, magnesium silicate, synthetic aluminum silicate, hardened oil, white beeswax, titanium oxide, microcrystalline cellulose, Macrogol 4000 and 6000, isopropyl myristate, calcium hydrogen phosphate, talc, etc.

Liquid formulations for oral administration may be illustrated as solutions for internal use, syrups, etc., and may include various excipients, such as humectants, sweeteners, fragrances, preservatives, etc., in addition to water and liquid paraffin which are simple diluents commonly used.

Formulations for parenteral administration may include sterile aqueous solutions, nonaqueous solvents, suspending agents, emulsions, lyophilization agents, and suppository agents. Nonaqueous solvent and suspending agent may include propylene glycol, polyethylene glycol, vegetable oil such as olive oil, and injectable esters such as ethyl oleate, etc. As bases for the suppository formulation, Witepsol, Macrogol, twin 61, cacao butter, laurin butter, glycerogelatin, etc. may be used. Injectable formulations may be prepared using aqueous solvents, such as physiological saline, Ringer's solution, etc., or non-aqueous solvents, such as vegetable oils, higher fatty acid esters (e.g., ethyl oleate), alcohols (e.g., ethanol, benzyl alcohol, propylene glycol, glycerin, etc.). In addition, the composition may include pharmaceutical carriers, including a stabilizer for preventing degeneration (e.g., ascorbic acid, sodium bisulfite, sodium pyrosulfite, BHA, tocopherol, EDTA, etc.), an emulsifier, a buffering agent for pH control, and a preservative for inhibiting microbial growth (e.g., phenylmercuric nitrate, thimerosal, benzalkonium chloride, phenol, cresol, benzylalcohol, etc.).

The pharmaceutical composition of the present disclosure may be administered in a pharmaceutically effective amount.

As used herein, the term “pharmaceutically effective amount” refers to an amount sufficient to treat diseases at a reasonable benefit/risk ratio applicable to medical treatment, and an effective dose level may be determined according to factors including a kind of subject, the severity, age, gender, a type of disease, activity of a drug, sensitivity to a drug, a time of administration, a route of administration, an excretion rate, duration of treatment, and agents to be simultaneously used, and other factors well known in the medical field. The composition of the present disclosure may be administered as an individual therapeutic agent or administered in combination with other therapeutic agents, and sequentially or simultaneously administered with existing therapeutic agents. In addition, the composition of the present disclosure may be administered once or several times. It is important to administer an amount capable of obtaining a maximum effect with a minimal amount without side effects in consideration of all of the factors, and the amount thereof may be easily determined by those skilled in the art. A preferred dosage of the composition of the present disclosure varies depending on a patient's condition and body weight, the severity of a disease, the form of a drug, and the route and duration of administration. For preferred effects, the composition of the present disclosure may be administered at a daily dose of 0.0001 mg/kg to 500 mg/kg, specifically, 0.001 mg/kg to 200 mg/kg. The dosage may be administered once a day or administered several times. The composition may be administered to various mammals such as mice, livestock, humans, etc. via various routes, and the mode of administration includes any method common in the art without limitation. The composition may be administered, for example, by oral, rectal or intravenous, intramuscular, subcutaneous, intrauterine dural or intracerebrovascular injection. Specifically, the composition may be orally administered, but is not limited thereto.

Further, the pharmaceutical composition of the present disclosure may be used in the form of medicinal products for animals as well as medicinal products applied for humans. Here, the animal is a concept including livestock and companion animals.

In the present disclosure, the composition of the present disclosure may be administered in combination with anti-cancer agents.

Alternatively, in the present disclosure, an anti-cancer agent may be loaded inside the nanocage. Specifically, the loading of the anti-cancer agent into the nanocage may be accomplished by culturing genetically engineered cells to produce the recombinant protein including the fusion protein including the programmed cell death protein 1 and the self-assembling protein in a cell culture medium in which the anti-cancer agent is dissolved, and adding the nanocage produced and isolated therefrom to a solvent, in which the anti-cancer agent is dissolved, followed by stirring. Specifically, a complex of divalent metal ions (e.g., Cu²⁺, Fe²⁺, and Zn²⁺) and the anti-cancer agent is formed, and then incubated with the prepared ferritin heavy chain nanocage in a buffer so that the complex of divalent metal ions and the anti-cancer agent may be loaded in the inner space of the prepared ferritin heavy chain nanocage. Alternatively, the anti-cancer agent may be loaded into the inner space through the disassemble-reassembly process of the ferritin heavy chain nanocage due to pH differences, and/or the anti-cancer agent may be loaded on the ferritin heavy chain nanocage by pore opening due to differences in ion concentration. Any method known in the art may be used without limitation, as long as it is a method of loading a compound inside a protein nanocage.

In the present disclosure, the anti-cancer agent may be, for example, taxane-based anticancer agents, statins, alkylating agents, platinum-based drugs, antimetabolites, antibiotics, vinca alkaloid anticancer agents, targeted therapy agents, antitumor immunotherapy agents, cancer vaccines, cell therapy agents, oncolytic virus, and combinations thereof, etc., and the anticancer therapy may be radiotherapy, photodynamic therapy, etc., but is not limited thereto, and may include all anticancer agents known in the art.

Examples of the taxane-based anticancer agents include paclitaxel, docetaxel, larotaxel, cabazitaxel, etc., but are not limited thereto.

The statins may be, but are not limited to, lipophilic statins. Examples of the lipophilic statins may include simvastatin, atorvastatin, lovastatin, fluvastatin, cerivastatin, pitavastatin, etc., but are not limited thereto.

Examples of the alkylating agents may include nitrogen mustard-based drugs, ethylenimine- and methyl melamine-based drugs, methyl hydrazine derivatives, alkyl sulfonate-based drugs, nitrosourea-based drugs, triazine-based drugs, etc., but are not limited thereto.

Examples of the platinum-based drugs may include any one or more selected from the group consisting of cisplatin, carboplatin, and oxaliplatin.

The antimetabolites may include folate antagonist-based drugs, purine antagonist-based drugs, pyrimidine antagonist-based drugs, etc., but are not limited thereto.

Examples of the antibiotics may include etoposide, topotecan, irinotecan, idarubicin, epirubicin, dactinomycin, doxorubicin (adriamycin), daunorubicin, bleomycin, mitomycin C, mitoxantrone, etc., but are not limited thereto.

Examples of the vinca alkaloid anticancer agents may include vincristine, vinblastine, vinorelbine, etc., but are not limited thereto.

Examples of the targeted therapy agents may include epidermal growth factor receptor (EGFR) targeted therapy agents, human epidermal growth factor receptor 2 (HER2) targeted therapy agents, B cell marker (CD20) targeted therapy agents, myeloid cell surface antigen (CD33) targeted therapy agents, cluster of differentiation 52 (CD52) targeted therapy agents, tumor necrosis factor receptor superfamily member 8 (CD30) targeted therapy agents, bcr-abl (breakpoint cluster region protein-Tyrosine-protein kinase)/c-Kit (tyrosine kinase receptor) targeted therapy agents, anaplastic lymphoma receptor tyrosine kinase (ALK) targeted therapy agents, antiangiogenics targeted therapy agents, mammalian target of rapamycin (mTOR) targeted therapy agents, cyclin-dependent kinase 4/6 (CDK4/6) targeted therapy agents, poly (ADP-ribose) polymerase (PARP) targeted therapy agents, proteasome inhibitors, tyrosine kinase antagonist agents, protein kinase C inhibitors, farnesyl transferase inhibitors, etc., but are not limited thereto.

Examples of the antitumor immunotherapy agents may include anti-programmed cell death protein 1 (PD-1), anti-programmed cell death protein 1 (PD-1) interaction inhibitors, anti-programmed cell death-ligand (PD-L) interaction inhibitors, cytotoxic T lymphocyte associated antigen 4 (CTLA4, CD152)/B7-1/B7-2 interaction inhibitors, cluster of differentiation 47 (CD47)/signal-regulatory protein (SIRP) interaction inhibitors, etc., but are not limited thereto.

Administration of the composition may be performed in combination with anticancer therapy. Examples of the anticancer therapy may include, for example, radiotherapy, photodynamic therapy, etc., but are not limited thereto. All anticancer therapies known in the art may be included.

Still another aspect of the present disclosure provides a protein nanocage produced by self-assembly of the fusion protein of the present disclosure.

Still another aspect of the present disclosure provides a drug delivery carrier including the nanocage of the present disclosure.

Still another aspect of the present disclosure provides a drug delivery system including the nanocage of the present disclosure.

As used herein, the term “drug delivery carrier” refers to any form of a carrier for further enhancing pharmacological activity of a loaded pharmacological component by loading a separate pharmacological component and moving to a lesion site or a target cell even though it does not have the pharmacological activity by itself or has the pharmacological activity.

As used herein, the term “drug delivery system” refers to a system, in which a drug delivery carrier is designed to exhibit physicochemical changes in response to stimuli such as pH, reduction, hypoxia, or reactive oxygen species.

The terms used in the above aspects are the same as described above.

Hereinafter, exemplary embodiments will be described in detail for better understanding of the present disclosure. However, the following exemplary embodiments are provided only for illustrating the present disclosure, but the scope of the present disclosure is not limited to the following exemplary embodiments. The exemplary embodiments of the present disclosure are provided to fully convey the concept of the present disclosure to those skilled in the art.

### EXAMPLE 1

### Preparation of Programmed Cell Death Protein 1 (PD-1)-Decorated Nanocages (NC) (PdNCs) and Physicochemical Characterization Thereof

Ferritin nanocages were prepared, the ferritin nanocages designed by surface engineering in order to display a PD-1 ecto-domain, which is capable of binding to programmed cell death-ligand 1 (PD-L1) and PD-L2.

In detail, wild-type human ferritin heavy chain (hFTN) nanocage (wtNC) and a monomeric form (SEQ ID NO: 1) of soluble PD-1 (sPD-1) of the ecto-domain of a murine PD-1 were synthesized by PCR from cDNA clones (Sino Biological Inc).

Meanwhile, a flexible linker (GSSGGSGSSGGSGGGDEADGSRGSQKAGVDE, SEQ ID NO: 14) consisting of an amino sequence, which links the human ferritin heavy chain (hFTN) and the ecto-domain of murine PD-1, was prepared by performing extension of PCR amplification with each primer. For purification by nickel affinity chromatography and steric hindrance avoidance, a histidine tag was linked to the ecto-domain of murine PD-1, which was then genetically incorporated into the C-terminal of the human ferritin heavy chain subunit with the prepared linker (FIG. 2A). The conformational flexibility of the C-terminal E-helix of ferritin subunit and the flexible glycine-rich linker was expected to provide sufficient accessibility of the ligands' binding capabilities.

Each DNA fragment of the synthesized wtNC, sPD-1, and PdNC including the linker between the human ferritin heavy chain and the ecto-domain of murine PD-1 was ligated with a pT7 vector to produce plasmids (pT7-wtNC, pT7-sPD-1, and pT7-PdNC). After the plasmids were constructed, the expression vectors were transformed into Escherichia coli (E. coli) strain BL21 (DE3), and the transformed cells were cultured with an ampicillin-resistant medium for selection. These cells were grown at 37° C. until the LB medium (Amp+) reached to OD600 of 0.5. After adding 1 mM isopropyl β-D-1-thiogalactopyranoside (IPTG), the protein expression was induced and incubated at 20° C. for 16 hr. The cells were obtained by centrifugation of the culture medium, resuspended, and homogenized with an ultrasonicator. The soluble proteins were purified using nickel-affinity chromatography and analyzed by sodium dodecyl sulfate-polyacrylamide gel electrophoresis (SDS-PAGE). To remove lipopolysaccharides, an endotoxin removal procedure was executed according to the protocol provided by the manufacturer (Thermo Scientific).

The recombinant protein produced from pT7-PdNC expressed by E. coli was self-assembled into a cage-like structure, and as a result, nanocages (PdNCs) decorated with 24 PD-1 on the surface of ferritin were prepared. As a result of SDS-PAGE analysis, the PdNCs were successfully biosynthesized in soluble proteins by using the E. coli expression system. The expressed and purified recombinant PdNCs appeared as a single band at 38.7 kDa (FIG. 2B).

Next, for dynamic light scattering (DLS) analysis of the sizes of the purified PdNCs and wtNCs, Zetasizer nano Zs (Malvern Instrument) was used. 1 μg of each of the purified samples was diluted in 1 mL of filtered PBS and analyzed under the following parameter conditions (temperature: 25° C., fixed angle: 173°, and refractive index of 1.450).

Further, a transmission electron microscopy (TEM) image of PdNCs was analyzed using a Tecnai F20 Cryo TEM (FEI Company), where 0.1 mg/mL of PdNCs was placed on a copper grid with a carbon film and negatively stained with a solution of uranyl acetate.

As a result, similar to the globular form of the wild-type ferritin nanocages (wtNCs), the PdNCs presented sphere-shaped particles with diameters of approximately 20 nm. The average diameter of the PdNCs was measured to be 21.95 nm, which was larger than that of the wtNCs (FIG. 2D). The TEM image of the PdNCs is as shown in FIG. 2C.

The above results indicate that PdNCs decorated with the PD-1 molecules on the surface of the nanocages with high density were successfully prepared.

### EXAMPLE 2

### Examination of Binding of PdNCs to PD-L1- and PD-L2-Upregulated Tumor Cells

To investigate the binding ability of PdNCs to the PD-L1- and PD-L2-expressed tumor cells, flow cytometry and fluorescence microscopy were performed.

In detail, CT26.CL25 colon tumor cells (ATCC), stably expressing both β-galactosidase (β-gal) and class I molecule H-2 Ld, were cultured in an RPMI-1640 (Welgene) medium with 10% fetal bovine serum (FBS) and 1% antibiotic-antimycotic (AA) (Gibco) at 5% CO₂ and 37° C. No mycoplasma contamination was detected in the cells used in this exemplary embodiment.

To examine the amounts of PD-L1 and PD-L2 on the cell membrane, 2×10⁵ CT26.CL25 cells were seeded onto a 100 pi dish and treated with 60 ng or 300 ng interferon-gamma (IFN-γ). On the next day, 5×10⁵ cells were pre-blocked with phosphate-buffered saline (PBS) containing 3% bovine serum albumin (BSA) at 4° C. for 15 min, and treated with a PD-L1 or PD-L2 antibody (R&D Science) at 4° C. for 30 min. After washing with Dulbecco's phosphate buffered saline (DPBS), the samples were stained with an Alexa Fluor 488-conjugated anti-goat IgG secondary antibody (Jackson ImmunoResearch) at 4° C. for 20 min. The PD-L1 or PD-L2 expression on the cell surface was measured using flow cytometry (n=3).

Further, to confirm the cell binding ability of PdNCs, 5×10⁵ CT26.CL25 cells (IFN-γ pretreated or not) were pre-blocked with 3% BSA/PBS at 4° C. for 1 hr. Then, they were incubated with 10 nM, 20 nM, or 40 nM of PdNCs, 40 nM of wtNCs, or PBS at 37° C. for 10 min. Afterwards, the cells were washed and labeled with an anti-ferritin heavy chain primary antibody (Abcam) and an Alexa Fluor 488-conjugated anti-goat IgG secondary antibody (Jackson ImmunoResearch) at 4° C. for 20 min. Then, the binding ability of NCs to each cell was detected via flow cytometry (n=4). For the fluorescence microscopic analysis of the cell binding ability of NCs, 3×10⁵ CT26.CL25 cells were seeded on the 35 pi confocal dish. The next day, the cells were fixed with 4% paraformaldehyde (PFA) for 5 min at room temperature, after washing with PBS. Afterwards, the cells were pre-blocked with 3% BSA/PBS at room temperature for 15 min and incubated with 40 nM of PdNCs, wtNCs, or a buffer at 4° C. for 20 min. The cells were then labeled with an anti-ferritin heavy chain primary antibody and an Alexa Fluor 488-conjugated anti-goat IgG secondary antibody at 4° C. for 20 min. Finally, the nuclei were stained with hoechest (H3570) diluted with 1% BSA at room temperature for 10 min (n=3).

As a result, as shown in FIG. 3A, wtNCs were able to bind to various cancer cells through transferrin receptor (TfR), and they showed a pattern of binding to CT26.CL25 cells (1.29 times the buffer control). However, PdNCs were more efficiently bound to CT26.CL25 cells in a concentration-dependent manner (8.80 times the buffer control), as compared to wtNCs. It was also confirmed that the amount of PdNCs bound to PD-L1 and PD-L2 overexpressed on CT26.CL25 cells was upregulated. This indicates that binding of PdNCs to the tumor cells may be increased even more for in vivo tumor microenvironment conditions.

In fluorescence microscopic images, CT26.CL25 cells treated with PdNCs also showed more fluorescence than that of wtNCs (FIG. 3B).

It is known that the PD-L expression on the tumor cell surfaces is often upregulated by the IFN-γ within the tumor microenvironment (TME). Consistently, the expression levels of PD-L1 and PD-L2 were significantly increased on the surface of the tumor cells treated with IFN-γ (FIG. 4). In a similar context, binding of PdNCs was significantly increased in IFN-γ-treated cells.

### EXAMPLE 3

### Examination of Affinity of PdNCs for PD-L1 and PD-L2 and Antagonistic Activity Thereof

To investigate the binding affinity and kinetics of PdNCs for PD-L1 and PD-L2, surface plasmon resonance (SPR) analysis was performed, and results were compared to the monomeric form of soluble PD-1 (sPD-1).

In detail, the binding affinities and kinetics of PdNCs or sPD-1 against murine PD-L1 (50010-M08H, Sino biological) or PD-L2 (50804-M08H, Sino Biological) were analyzed by using an SPR instrument (SR7500 DC, Buffalo, Reichert Inc.). Before the analysis, an SR7000 gold sensor slide (Reichert Inc.) was stabilized with a running buffer. Then, the murine PD-L1 or PD-L2 in a sodium acetate buffer (pH=5) was immobilized on the surface of a dextran chip. To prevent non-specific binding of the analyte, the chip was coated with BSA (15 nM). PdNCs and sPD-1 were suspended in Tris buffer (20 mM, pH=7.4), which was the same as the running buffer. PdNCs (2.5-500 nM) or sPD-1 (1-50 μM) were subjected to two-point serial dilution before the analysis. Each sample was allowed to flow at a continuous rate (50 μL/min). The bindings of the samples and ligands were analyzed in real time. The titration sensorgrams were analyzed using a 1:1 binding model of Langmuir (A+B⇔AB) by utilizing Scrubber 2.0 (BioLogic Software) and KaleidaGraph (Synergy Software).

As a result, as shown in FIG. 5A, it was confirmed that both PdNCs and sPD-1 were bound to PD-L1 and PD-L2 in a concentration-dependent manner, but sPD-1 bound to PD-L1 and PD-L2 has a low affinity. PdNCs were bound to PD-L1 and PD-L2 with nanomolar and sub-nanomolar affinities. Higher association rates (kₐ) and lower dissociation rates (k_{d}) of PdNCs than sPD-1 were observed for both ligands. An equilibrium dissociation constant (K_{D}) for PdNCs was decreased by 1057 times for PD-L1 and 647 times for PD-L2 in comparison to sPD-1. Thus, PD-1 on the surfaces of the PdNCs was readily recognized by ligands with an enhanced avidity effect.

The above results indicate that PdNCs may provide enhanced antagonistic efficiency.

Next, in vitro antagonistic activity of PdNCs was demonstrated using a bioluminescent PD-1/PD-L1 blockade bioassay.

In detail, CHO-K1 cells expressing murine PD-L1 and a protein that was designed to activate cognate TCRs were treated with PdNCs, sPD-1, or wtNCs. Continuously, Jurkat cell lines expressing PD-1, TCR, and nuclear factor activated T cell (NFAT)-inducible luciferase, which replaced primary T cells, were added and co-cultured for 6 hr. Relative bioluminescence (RLU) was detected by using a multi-detection micro plate reader (Spectramax i3x, R&D mate). The half maximal effective concentrations (EC50) were calculated by using the Hill equation from the experimental data (n=3).

As a result, treatment of Jurkat T cells with the low dosage of PdNCs successfully increased TCR activation and NFAT-mediated luciferase expression through interfering of the PD-1/PD-L1 axis (FIG. 5B). There was no signaling change for the wtNC-treated Jurkat T cells (FIG. 6), and the PdNC- and sPD-1-treated cells exhibited dose-dependent TCR activation signaling.

Meanwhile, there was substantial increase in the NFAT-mediated luciferase expression in the Jurkat T cell that was observed with treatment of a higher sPD-1 dosage. In particular, consistent with the results of the binding affinity analysis, PdNCs exhibited a half maximal effective concentration (EC50) of 761.3 pM, which is 624 times higher than that of sPD-1 (457.3 nM).

The above results indicate that PdNC substantially improved their recognition due to their enhanced affinity, suggesting that it may serve as an antagonistic agent for anti-cancer immunotherapy.

### EXAMPLE 4

### Examination of Accumulation and Induction of DC-Mediated T Cell Activation by PdNC in Tumor-Draining Lymph Node (TDLN)

It is known that dendritic cell (DC)-mediated T cell priming and activation in lymph nodes (LNs) are crucial to achieving efficient anti-tumor immunity (Siddiqui, K., et al., Immunity, 50 (2019) 195-211.e110). In particular, through intravital imaging of LNs, an antigen-presenting DC interacts with 500-5000 T cells per hour, and the interaction between the DCs and cognate T cell lasts approximately for a day after immunization (R. Obst, Front. Immunol., 6 (2015), p. 563). Thus, the delivery efficiency of PdNCs against TDLNs at 1 hr, 6 hr, 18 hr, and 24 hr was investigated.

In detail, 48 μM of sPD-1, 2 μM of wtNC, or 2 μM of PdNC were mixed with 48 μM of Cyanine5.5 (Cy5.5) NHS ester (Bioacts) and incubated at 4° C. overnight to prepare Cy5.5 NHS ester-labeled PdNC, sPD-1, or wtNC. Free cy5.5 unconjugated with NC was separated using an Ultra Centrifugal Filter (Milipore). Before being injected into mice, the Cy5.5-conjugated samples were analyzed with a microplate reader (GloMax Discover, Promega) to match the fluorescence intensity between the samples.

Meanwhile, 8-week BALB/c white and BALB/c nude mice (OrientBio) were raised in pathogen-free conditions. 5×10⁵ CT26.CL25 cells were inoculated into the left flank of BALB/c nude mice. CT26.CL25 tumor cells were implanted into BALB/c nude mice. When the tumor size reached approximately 200 mm³, Cy5.5 NHS ester-labeled PdNC, sPD-1, wtNC or the free dye (Cy5.5) were intratumorally injected. To examine relative fluorescence signal intensity of LN to tumors at 1 hr, 6 hr, 18 hr, or 24 hr after injection, LNs and tumor tissues were resected at the indicated time-point. The resected TDLNs and tumor tissues were visualized with an IVIS spectrum (IVIS® Lumina Series III, Caliper Life Sciences). The relative fluorescence intensity was calculated as the relative intensity of TDLNs to the tumor tissue (n=4-5).

As a result, intratumorally injected PdNCs were rapidly drained and accumulated in TDLNs at 1 hr post injection (FIG. 7A). After 6 hr and 18 hr post injection, although the fluorescence intensity in TDLNs of the wtNC-treated mice gradually increased due to its size, the fluorescence intensity in TDLNs of the PdNC-treated mice are the highest at all time-points. In particular, at 24 hr, a stronger fluorescence intensity in TDLNs of the PdNC-treated mice was observed than in any other group.

The above results indicate that the PdNCs efficiently reached and accumulated in the TDLNs.

Next, whether the observed efficient TDLN-targeting ability of PdNCs can promote the anti-tumor immune response in the TDLNs was assessed using the same CT26.CL25 syngeneic tumor mouse model.

In detail, BALB/c white mice were inoculated with 1×10⁶ CT26.CL25 cells in the left flank. On day 6 after inoculation, when the tumor size reached approximately 70 mm³, PdNCs (23.7 mg/kg), wtNCs (13.1 mg/kg), sPD-1 (10.0 mg/kg), or PBS was injected into the tumor (n=6-10). The tumor size was checked with a caliper every 3 days and calculated by applying the formula (width²×length)/2. The tumor-free mice were re-injected in the opposite site of the primary tumor with 1×10⁶ CT26.CL25 tumor cells four weeks later after complete remission.

To analyze in vivo DC maturation, the CT26.CL25 tumor-bearing mice were injected with PdNCs, wtNCs, sPD-1, or PBS. On day 3 after injection, TDLNs were extracted and turned into a single-cell suspension using a syringe plunger on day 9 after CT26.CL25 tumor inoculation. After preparing pellets by performing centrifugation, red blood cells (RBCs) were lysed with a lysis buffer (Biolegend). The relative mean fluorescence intensity (MFI) (relative MFI to the control) of CD40 or CD86 on CD11c+ DC from the single-cell suspensions was analyzed via flow cytometry, which was achieved by using CD11c (APC, Biolegend)+CD40 (PE, Biolegend) or CD86 antibody (PE, Biolegend). A rat IgG2a isotope (PE, Biolegend) and an Armenian Hamster IgG isotype antibody (APC, Biolegend) were used as controls (n=4-5).

To verify the antigen-experienced T cells, the TDLN single-cell suspensions were stained with ant-CD8 (FITC, Biolegend) and anti-CD44 (PE, Biolegend) antibodies. The relative MFI was analyzed by flow cytometry (n=4-5).

As a result, the PdNC treatment induced increases in the co-stimulatory molecules (CD40 and CD86) on DCs in the TDLNs, enhancing the DC maturation (FIG. 7B).

Furthermore, as shown in FIG. 7C, the PdNC treatment increased CD44+ in the CD8+ T cells, wherein CD44+ is a marker for antigen-experienced T cells.

This result indicates that PdNCs efficiently blocked the PD-1/PD-Ls interaction, thereby enhancing DC maturation and T cell activation.

Next, to confirm whether tumor-specific immunity occurred, the single-cell suspensions of TDLNs were treated with tumor-specific gp70 and β-gal peptide, which are CT26.CL25 tumor cell-associated antigens.

In detail, 5×10⁵ cells of the single-cell suspension of TDLN were seeded in a 96-well plate and incubated with 5 μg/mL of gp70 or β-gal. After 24 hr, the supernatant was collected and used to evaluate the tumor-specific immune response with an IFN-γ ELISA Kit (R&D Systems) (n=3-4).

As a result, as expected, PdNC treatment significantly increased the IFN-γ secretion against gp70 and β-gal, unlike wtNCs and sPD-1 (FIG. 7D).

Next, it is well known that PD1/PD-Ls interaction limits T cell function during priming or activation (S. J. P. Blake, et al., PLoS One, 10 (2015), Article e0119483), and therefore, to investigate whether PdNCs potentiate the cross-prime abilities of DCs, ex vivo cross-priming assays were performed by measuring the IFN-γ secretion from the supernatant of the co-cultured plates of CD8+ splenocytes with CD11c+ cells from TDLNs.

In detail, CD11c+ or CD8+ cells were isolated using CD11c+ or CD8 MicroBeads (Miltenyi Biotec) from the single-cell suspension of TDLNs or spleen. Then, 3×10⁴ of CD11c+ cells and 1.2×10⁵ of CD8+ cells were seeded in a 96-well plate with 100 ng/mL of IL-2. After 48 hr, the supernatant was used to investigate the amount of IFN-γ with IFN-γ ELISA Kit (R&D Systems) (n=4-5).

As a result, the PdNC-treated group showed remarkable IFN-γ secretion, as compared to other groups (FIG. 7E).

These results suggest that the enhanced delivery and antagonistic ability of PdNCs potentiates the activated DC-mediated anti-tumor T cell immune responses in TDLNs.

### EXAMPLE 5

### Tumor Growth Inhibitory Effect of PdNC

The enhanced tumor growth inhibitory effect based on the anti-tumor immunity according to PdNC treatment was evaluated. When the average size of the tumor reached 70 mm³, the mice were treated with PdNCs, sPD-1, wtNCs, or a buffer via an intratumoral single injection.

As a result, as shown in FIGS. 8A and 9, the PdNC-treated group dramatically reduced the tumor growth in comparison to other groups. The PdNCs suppressed the tumor volumes by 75%, whereas the 24 times higher molar doses of the sPD-1 resulted in no significant reduction of the tumor volume. Further, complete tumor regression was observed in approximately 33% (3 of 9) of the PdNC-treated group with only a single injection.

Next, to analyze in vivo potential toxicity, CT26.CL25 bearing 7 week-old BALB/c white mice were administered with PdNCs, wtNCs, sPD-1, or PBS. After 48 hr later of injection, the body weights of the mice of each group were measured, and the tumor tissue, liver, lung, and kidney were extracted and fixed with formalin (Sigma) and embedded in paraffin blocks. For hematoxylin and eosin (H&E) staining, the paraffin blocks were cut into 4 μg-thick sections, deparaffinized by treatment with xylene for 1 hr, and rehydrated by treatment with EtOH for 5 min. The slides were then stained with hematoxalin for 10 min and eosin for 30 sec. Deionized or tap water was used in washing. Then, tissue damage was analyzed with fluorescence microscopy (Olympus BX51) (n=2-5, different fields for each image).

As a result, there was no decrease in the body weight of the mice of all groups, including in those treated with PdNCs, and there were no significant differences between the groups (FIG. 10). H&E stained images of major organs including liver, lung, and kidney from the PdNC-treated mice exhibited no differences compared to those in the buffer control group, indicating no significant toxicity due to PdNCs (FIG. 11).

Next, at the end of the experiment, the tumor tissues from the treated mice were resected and analyzed for an anti-tumor immune response.

The tumors harvested on day 9 after the CT26.CL25 tumor inoculation were turned into single-cell suspensions by using a tumor dissociation kit and a gentleMACS machine (MACS, Miltenyi Biotec). RBCs were lysed with a lysis buffer, and the apoptotic cells were removed with a dead cell removal kit. The suspensions were sorted with CD8 specific magnetic beads to isolate the CD8+ cells. The isolated cells were treated with an anti-CD8α (FITC, Biolegend) antibody, an anti-mouse IFN-γ (APC, Biolegend) antibody, or an anti-mouse Ki-67 antibody (PE, Biolegend) (n=3-4). To analyze DC infiltration in TME, the suspensions were labeled with an anti-mouse CD45.2+ (PerCP Cy5.5, Biolegend) antibody and an anti-mouse CD11c+ (APC, Biolegend) antibody. Afterwards, they were analyzed by flow cytometry (n=4-5).

As a result, as shown in FIG. 8B, the PdNC treatment increased ki67 and IFN-γ expression in CD3+ CD45.2+ CD8+ T cells in TME, which are markers for the proliferation and effector functions of T cells, suggesting activation of the T cell immunity in TME.

Next, tumor tissues were extracted on day 21 after inoculation of CT26.CL25 tumor into CT26.CL25 mice model, and embedded into an OCT compound (Leica Biosystems). After creating a frozen block, the samples were sectioned by a rotary microtome for CD8+ cell staining. The sections were pre-blocked with 3% BSA/PBS for 2 hr. Then, the frozen samples were incubated with CD8α (BD Pharmigen) antibodies at 4° C. overnight. Next day, the sections were washed and incubated with an Alexa Fluor 488-conjugated secondary antibody (Jackson ImmunoResearch). A rat IgG2a isotype control antibody (Jackson ImmunoResearch) was used as a control. The labeled CD8+ T cells in tumor tissues were detected using a fluorescence microscope (Nikon eclipse Ti). The number of T cells infiltrated into tumor tissues per mm² was calculated using ImageJ (n=3-5, different fields for each image).

As a result, PdNCs-treated group induced a significant increase in the tumor-infiltrating CD8+ T cells and DCs in TME in comparison to other groups (FIGS. 8C-8E). In particular, the tumor-infiltrating CD8+ T cells in the PdNC-treated mice demonstrated a significant increase (4.64 times the buffer control) (FIG. 8D).

Further, when the PdNC-treated tumor-free mice were injected with the tumor cells derived from the tumor tissue at 21 days after inoculation of CT26.CL25 tumor into the CT26.CL25 mouse model, there was no tumor growth, indicating that a specific immunologic memory was formed (FIG. 8F).

Taken together, the above results confirmed that PdNCs can elicit an anti-tumor response by upregulating the DC-mediated T cell activation in TME and TDLNs, which leads to the formation of a desirable anti-tumor immunity in the tumor microenvironment.

According to the results of the exemplary embodiments, the PD-1-decorated nanocage (PdNC) of the present disclosure may block PD-1 and PD-L signaling and may induce anti-tumor immunity activation at two immune checkpoints of TME (effector phase) and TDLN (innate phase), thereby increasing the adaptability of PD-1 and PD-L blockade-based therapy. Accordingly, it may be applied to various kinds of cancer therapies.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are therefore intended to be embraced by the claims.

### Effect of the Invention

A programmed cell death protein 1 (PD-1)-decorated nanocage (PdNC) of the present disclosure may block PD-1 and programmed cell death-ligand (PD-L) signaling and may induce anti-tumor immunity activation at two immune checkpoints of tumor microenvironment (TME) (effector phase) and tumor-draining lymph node (TDLN) (innate phase), thereby increasing the adaptability of PD-1 and PD-L blockade-based therapy. Accordingly, it may be applied to various kinds of cancer therapies.

What is claimed is:

1. A pharmaceutical composition for preventing or treating cancer, the pharmaceutical composition comprising, as an active ingredient, nanocages formed by self-assembly of a fusion protein including a programmed cell death protein 1 and a self-assembling protein.

2. The pharmaceutical composition of claim 1, wherein the nanocages induce anti-tumor immunity activation at two immune checkpoints of an effector phase and an innate phase.

3. The pharmaceutical composition of claim 2, wherein the anti-tumor immunity activation at the effector phase occurs in the tumor microenvironment (TME).

4. The pharmaceutical composition of claim 2, wherein the anti-tumor immunity activation at the innate phase occurs in the tumor-draining lymph node (TDLN).

5. The pharmaceutical composition of claim 2, wherein the anti-tumor immunity activation is dendritic cell-mediated tumor-specific T cell activation.

6. The pharmaceutical composition of claim 1, wherein the nanocages block any one or more signals selected from a programmed cell death protein 1 and a programmed cell death-ligand.

7. The pharmaceutical composition of claim 1, wherein the self-assembling protein is any one or more selected from the group consisting of ferritin, small heat shock protein (sHsp), vault, P6HRC1-SAPN, M2e-SAPN, MPER-SAPN, virus capsid proteins, and bacteriophage capsid proteins.

8. The pharmaceutical composition of claim 7, wherein the self-assembling protein is ferritin.

9. The pharmaceutical composition of claim 8, wherein the ferritin is any one or more selected from a ferritin heavy chain protein and a ferritin light chain protein.

10. The pharmaceutical composition of claim 9, wherein the ferritin is a ferritin heavy chain protein.

11. The pharmaceutical composition of claim 7, wherein the virus capsid protein and the bacteriophage capsid protein are any one or more selected from the group consisting of a bacteriophage MS2 capsid protein, a bacteriophage P22 capsid protein, a Qβ bacteriophage capsid protein, a CCMV capsid protein, a CPMV capsid protein, an RCNMV capsid protein, an ASLV capsid protein, an HCRSV capsid protein, an HJCPV capsid protein, a BMV capsid protein, an SHIV capsid protein, an MPV capsid protein, an SV40 capsid protein, an HIV capsid protein, an HBV capsid protein, an adenovirus capsid protein, and a rotavirus VP6 protein.

12. The pharmaceutical composition of claim 1, wherein the programmed cell death protein 1 and the self-assembling protein are linked via a linker.

13. The pharmaceutical composition of claim 1, wherein the programmed cell death protein 1 includes an amino acid sequence of SEQ ID NO: 1.

14. The pharmaceutical composition of claim 1, wherein the self-assembling protein includes any one or more amino acid sequences selected from the group consisting of SEQ ID NOS: 3 to 13.

15. The pharmaceutical composition of claim 12, wherein the linker includes an amino acid sequence of SEQ ID NO: 14.

16. A method of preventing or treating cancer, the method comprising the step of administering, to an individual excluding humans, a pharmaceutical composition including, as an active ingredient, nanocages formed by self-assembly of a fusion protein including a programmed cell death protein 1 and a self-assembling protein.

17. The method of claim 16, wherein the composition is administered in combination with an anti-cancer agent.

18. The method of claim 16, wherein the anti-cancer agent is loaded inside the nanocages.

19. The method of claim 17, wherein the anti-cancer agent is any one or more selected from the group consisting of taxane-based anticancer agents, statins, alkylating agents, platinum-based drugs, antimetabolites, antibiotics, vinca alkaloid anticancer agents, targeted therapy agents, antitumor immunotherapy agents, cancer vaccines, cell therapy agents, oncolytic virus, and combinations thereof.

20. The method of claim 19, wherein the taxane-based anticancer agent is any one or more selected from the group consisting of paclitaxel, docetaxel, larotaxel, and cabazitaxel.

21. The method of claim 19, wherein the statin is a lipophilic statin.

22. The method of claim 21, wherein the lipophilic statin is any one or more selected from the group consisting of simvastatin, atorvastatin, lovastatin, fluvastatin, cerivastatin, and pitavastatin.

23. The method of claim 19, wherein the alkylating agent is any one or more selected from the group consisting of nitrogen mustard-based drugs, ethylenimine- and methyl melamine-based drugs, methyl hydrazine derivatives, alkyl sulfonate-based drugs, nitrosourea-based drugs, and triazine-based drugs.

24. The method of claim 19, wherein the platinum-based drug is any one or more selected from the group consisting of cisplatin, carboplatin, and oxaliplatin.

25. The method of claim 19, wherein the antimetabolite is any one or more selected from the group consisting of folate antagonist-based drugs, purine antagonist-based drugs, and pyrimidine antagonist-based drugs.

26. The method of claim 19, wherein the antibiotic is any one or more selected from the group consisting of etoposide, topotecan, irinotecan, idarubicin, epirubicin, dactinomycin, doxorubicin (adriamycin), daunorubicin, bleomycin, mitomycin C, and mitoxantrone.

27. The method of claim 19, wherein the vinca alkaloid anticancer agent is any one or more selected from the group consisting of vincristine, vinblastine, and vinorelbine.

28. The method of claim 19, wherein the targeted therapy agent is any one or more selected from the group consisting of epidermal growth factor receptor (EGFR) targeted therapy agents, human epidermal growth factor receptor 2 (HER2) targeted therapy agents, B cell marker (CD20) targeted therapy agents, myeloid cell surface antigen (CD33) targeted therapy agents, cluster of differentiation 52 (CD52) targeted therapy agents, tumor necrosis factor receptor superfamily member 8 (CD30) targeted therapy agents, bcr-abl (breakpoint cluster region protein-Tyrosine-protein kinase)/c-Kit (tyrosine kinase receptor) targeted therapy agents, anaplastic lymphoma receptor tyrosine kinase (ALK) targeted therapy agents, antiangiogenics targeted therapy agents, mammalian target of rapamycin (mTOR) targeted therapy agents, cyclin-dependent kinase 4/6 (CDK4/6) targeted therapy agents, poly (ADP-ribose) polymerase (PARP) targeted therapy agents, proteasome inhibitors, tyrosine kinase antagonist agents, protein kinase C inhibitors, and farnesyl transferase inhibitors.

29. The method of claim 19, wherein the antitumor immunotherapy agent is any one or more selected from the group consisting of anti-programmed cell death protein 1 (PD-1)/anti-programmed cell death-ligand (PD-L) interaction inhibitors, cytotoxic T lymphocyte associated antigen 4 (CTLA4, CD152)/B7-1/B7-2 interaction inhibitors, and cluster of differentiation 47 (CD47)/signal-regulatory protein (SIRP) interaction inhibitors.

30. The method of claim 16, wherein the composition is administered in combination with anticancer therapy.

31. The method of claim 30, wherein the anticancer therapy is any one or more selected from the group consisting of radiotherapy and photodynamic therapy.

32. A protein nanocage formed by self-assembly of the fusion protein of claim 1.
